(19) United States
(12) Patent Application Publication
(10) Pub. No.: US 2026/0215809 A1
(43) Pub. Date: Jul. 30, 2026

(54) SPRING LOADED LOCKING AND FOLDING SCALPEL

(71) Applicant: Hawk Designs Inc., Idaho City, ID (US)

(72) Inventor: Gavin Dan HAWK, Idaho City, ID (US)

(21) Appl. No.: 19/039,255

(22) Filed: Jan. 28, 2025

(51) Int. Cl. A61B 17/3213; A61B 17/00; B26B 5/00

(52) U.S. Cl. CPC A61B 17/3213; A61B 2017/00836; B26B 5/00

(57) ABSTRACT

A foldable knife that includes a first handle, a second handle pivotably coupled to the first handle, and a blade attached to the second handle and not to the first handle. The foldable knife further includes a locking mechanism configured to secure the foldable knife in a first configuration and a second configuration. The blade is partially concealed in the first configuration and the blade is exposed in the second configuration. In the first configuration, the first handle and the second handle are disposed in parallel planes. In the second configuration, the first handle and the second handle are disposed in different parallel planes.

### TECHNICAL FIELD

The present disclosure relates generally to the field of foldable knives. More particularly, some embodiments relate to foldable knife with a replaceable scalpel blade.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a side view of a foldable knife in a closed configuration according to one embodiment of the present disclosure.

FIG. 2 illustrates a side view of a foldable knife in a closed configuration according to one embodiment of the present disclosure.

FIG. 3 illustrates a side view of a scalpel blade for a foldable knife according to one embodiment of the present disclosure.

FIG. 4 illustrates a top view of a foldable knife in a closed configuration according to one embodiment of the present disclosure.

FIG. 5 illustrates a cross-sectional view of a foldable knife with a locking mechanism in a first locked position and a slider of the locking mechanism in a first position, according to one embodiment of the present disclosure.

FIG. 6 illustrates a cross-sectional view of the foldable knife of FIG. 5 with the slider of the locking mechanism in a second position.

FIG. 7 illustrates a cross-sectional view of the foldable knife of FIG. 5 with the locking mechanism in a second locked position and the slider in the first position.

FIG. 8 illustrates a side view of a foldable knife in an open configuration, according to one embodiment of the present disclosure.

FIG. 9 illustrates a side view of a foldable knife in an open configuration according to one embodiment of the present disclosure.

FIG. 10 illustrates a user gripping a foldable knife in a closed configuration according to one embodiment of the present disclosure.

FIG. 11 illustrates a user gripping the foldable knife in an open configuration according to one embodiment of the present disclosure.

FIG. 12 illustrates a user gripping a foldable knife in an open configuration according to one embodiment of the present disclosure.

FIG. 13 illustrates a user gripping a foldable knife in an open configuration according to one embodiment of the present disclosure.

FIG. 14 illustrates a user gripping a foldable knife in an open configuration according to one embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive).

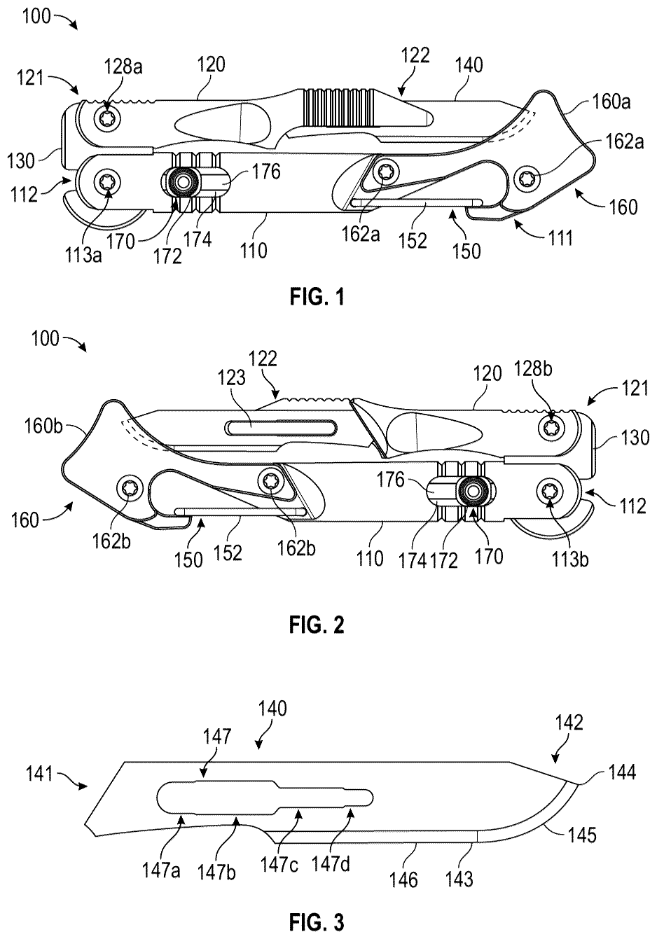

The present disclosure is directed to a foldable knife 100 that comprises a first or closed configuration and a second or open configuration. The foldable knife 100 is moveable between the first configuration and the second configuration. FIGS. 1 and 2 illustrate opposite side views of a foldable knife 100 in a first configuration according to one embodiment of the present disclosure. The foldable knife 100 comprises two handles, a first handle 110 and a second handle 120 that are pivotably coupled to each other. The first handle 110 comprises a first end 111 and a second end 112. The second handle 120 comprises a first end 121 and a second end 122.

In the illustrated embodiment, the foldable knife 100 includes a linkage 130 that pivotably couples the first handle 110 to the second handle 120. The linkage 130 is rigidly attached to the second handle 120. In some embodiments, the linkage 130 may be integral with the second handle 120. In the illustrated embodiment, the second end 112 of the first handle 110 is attached to the linkage 130 via fasteners 113a, 113b. In some embodiments, the first handle 110 is attached to the linkage 130 with a single fastener. The first end 121 of the second handle 120 is attached to the linkage 130 via a fastener 128, 128b. In some embodiments, the second handle 120 is attached to the linkage 130 with a single fastener. The second handle 120 pivotably rotates relative to the first handle 110 along an axis of rotation. The axis of rotation may coincide with the fasteners 113a, 113b that attach the first handle 110 to the linkage 130.

The foldable knife 100 further comprises a blade 140 that is attached directly to the second handle 120, but not to the first handle 110. FIG. 3 illustrates a detailed version of the blade 140. In the illustrated embodiment, the blade 140 is a scalpel. However, the foldable knife 100 is not limited to the illustrated embodiment of the blade 140. Various types of blades and/or scalpel blades may be used in conjunction with the foldable knife 100. The illustrated blade 140 is merely one type of scalpel blade 140 that may be used in conjunction with the foldable knife 100.

The blade 140 comprises a first end 141 and a second end 142. The blade 140 comprises a cutting edge or cutting element 143 that extends from a tip 144 at the second end 142 of the blade 140, a curved portion 145, and a straight portion 146. In the illustrated embodiment, the cutting element 143 does not extend to the first end 141 of the blade 140.

The blade 140 further comprises an aperture 147 that varies in size along a length of the aperture 147. For example, in the illustrated embodiment, the aperture 147 comprises four different size portions. A first portion 147a, a second portion 147b, a third portion 147c, and a fourth portion 147d. The second portion 147b has the greatest height, the first portion 147a has the second greatest height, the third portion 147c has the third greatest height, and the fourth portion 147d has the smallest height.

The blade 140 is removably attachable to the second handle 120 via the aperture 147. As illustrated in FIG. 2, the second end 122 of the second handle 120 comprises a projection 123 that is sized to receive the aperture 147 of the blade 140. The size of the projection 123 is similar in size to the second portion 147b of the aperture 147 of the blade 140. While not illustrated, the projection 123 comprises a slot behind the projection 123 that enables a user to slide the blade 140 on the projection 123 toward the first end 121 so that the third portion 147c and the fourth portion 147d of the aperture 147 are configured to engage with the slot and secure the blade 140 to the second end 122 of the second handle 120. The blade 140 may be removed by sliding the blade 140 in the opposite direction to remove the blade 140 from the second end 122 of the second handle 120.

In some embodiments, the first handle 110 and/or the second handle 120 may be formed primarily of titanium. In some embodiments, the blade may be formed primarily of stainless steel. In some embodiments, the projection 123 and a portion of the second handle 120 contacting the blade 140 may be formed of a material other than titanium, to reduce galling.

The first handle 110 further comprises a clip mechanism 150. The clip mechanism 150 comprises a biased clip 152. The biased clip 152 may be push into an aperture 154 of the first handle 110 enabling the clip mechanism 150 to be coupled to something, such as a belt loop or keychain. After the clip mechanism 150 is coupled, the biased clip 152 may be released and the biased clip 152 is biased to its original position illustrated in FIGS. 1 and 2, enabling the foldable knife 100 to be coupled to something.

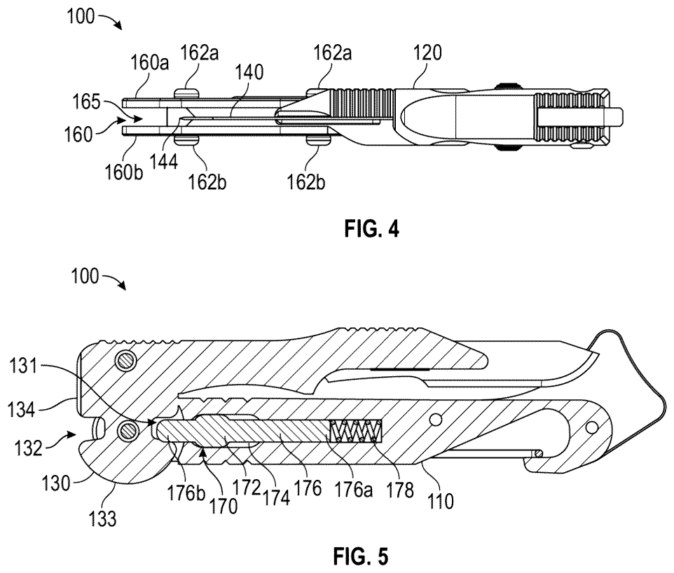

The first handle 110 further comprises a protective sleeve 160 for receiving a portion of the blade 140. In the illustrated embodiment, the protective sleeve 160 comprises a pair of guards 160a and 160b. Each guard 160a, 160b is secured to the first handle 110 via fasteners 162a, 162b and are attached to opposite sides of the first handle 110. FIG. 4 illustrates a top view of the foldable knife 100 in which the pair of opposing guards 160a, 160b form a slot 165 in between to receive the tip 144 of the blade 140. As illustrated in FIG. 4, a width of the blade 140 is thinner than a width of the first handle 110 and a width of the second handle 120.

As illustrated in the FIGS. 1 and 2, the tip 144 and the curved portion 145 are disposed within the protective sleeve 160 when the foldable knife 100 is in the first or closed configuration. The tip 144 and the curved portion 145 are illustrated in broken lines so show that tip 144 and the curved portion 145 are concealed in the first configuration. The cutting element 143 of the blade 140 is oriented towards the first handle 110 in the first configuration. In some embodiments, the blade 140 is partially concealed in the first configuration. In some embodiments, the cutting element 143 of the blade 140 is partially concealed in the first configuration. As illustrated in FIGS. 1 and 2, the straight portion 146 of the cutting element 143 is not enclosed within the protective sleeve 160. In some embodiments, the cutting element 143 is totally enclosed within the protective sleeve 160. In some embodiments, the blade 140 is totally enclosed within the protective sleeve 160.

The foldable knife 100 comprises a locking mechanism 170 that is configured to selectively secure the foldable knife 100 in the first configuration and in the second configuration. In other words, the user may selectively decide which configuration to use for either storage or use and may move the foldable knife 100 from the first configuration to the second configuration and vice versa. In the illustrated embodiment, the locking mechanism 170 comprises a slider 172 disposed within a slot 174 and a rod 176 that is configured to engage with the linkage 130.

FIGS. 1-2 and 4-6 illustrate the foldable knife 100 in the first configuration and FIGS. 7-9 illustrated the foldable knife 100 in the second configuration. As discussed above, the second handle 120 rotates with linkage 130 about an axis of rotation to transition the foldable knife 100 from the first configuration to the second configuration. The axis of rotation may coincide with fasteners 113a, 113b. The second handle 120 rotates about 180 degrees relative to the first handle 110 from the first configuration to the second configuration. In the first configuration, the first handle 110 and the second handle 120 are disposed in parallel planes and not in the same plane. The foldable knife 100 may have a substantially rectangular shape in the first configuration. As discussed above, in the first configuration, the tip 144 the curved portion 145 of blade 140 are concealed within the protective sleeve 160 of the foldable knife 100.

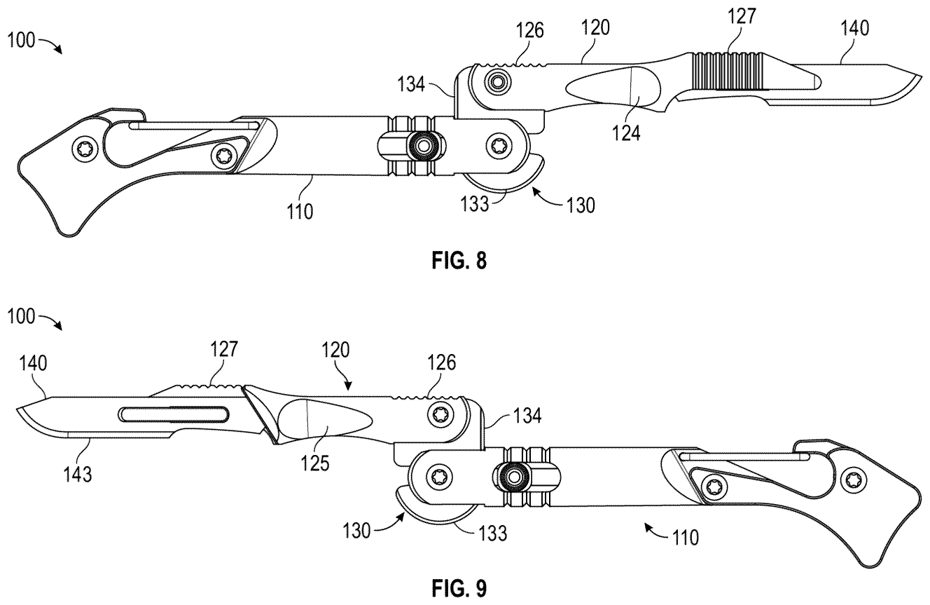

In the second configuration, the first handle 110 and the second handle 120 are disposed is parallel planes and not in the same plane. In the second configuration, the second handle 120 is disposed in a different plane than in the first configuration and the first handle 110 is disposed in the same plane as the first configuration. In other words, the two parallel planes are different in the second configuration than the first configuration as a result of the movement of the second handle 120 relative to the first handle 110. The longitudinal distance (i.e., along the length of the handle) from the second end 122 of second handle 120 to the first end 111 of the first handle 110 in the second configuration is greater than the longitudinal distance from the second end 122 of second handle 120 to the first end 111 of the first handle 110 in the first configuration. The foldable knife 100 in the second configuration has a stepped shape. Accordingly, a longitudinal distance from the second end 122 of second handle 120 to the first end 111 of the first handle 110 in the second configuration is greater than the longitudinal distance from the second end 122 of second handle 120 to the first end 111 of the first handle 110 in the first configuration. In the second configuration, as illustrated in FIGS. 8 and 9, the blade 140 and the cutting element 143 are exposed.

In the first configuration of the foldable knife 100, the second handle 120 is disposed on a first longitudinal side of the first handle 110. In the second configuration of the foldable knife 100, the second handle 120 is disposed on a second longitudinal side of the first handle 110, opposite the first longitudinal side of the first handle 110.

In the first configuration of the foldable knife 100, the first handle 110 and the second handle 120 overlap a longitudinal length of the second handle 120. In the second configuration of the foldable knife 100, the first handle 110 and the second handle 120 only overlap at a pivot connection between the first handle 110 and the second handle 120 as illustrated in FIGS. 8 and 9.

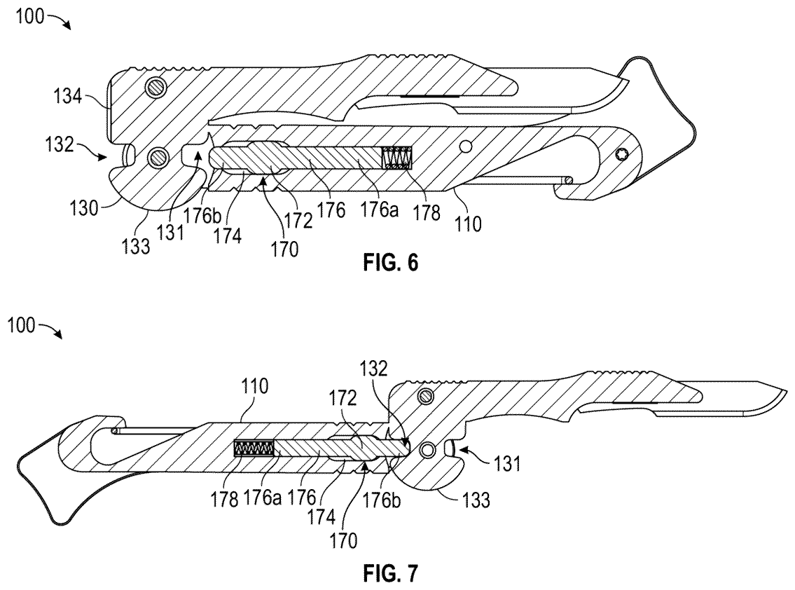

FIGS. 5-7 illustrate cross-sectional views of the foldable knife 100. Specifically, FIGS. 5-7 illustrate the various components of the locking mechanism 170. The locking mechanism 170 may be a biasing locking mechanism. FIG. 5 illustrates the slider 172, the slot 174, the rod 176, and a biasing member 178 of the locking mechanism 170. The rod 176 and the biasing member 178 are disposed within the first handle 110. While FIGS. 5-7 illustrate the biasing member 178 as a spring, the present disclosure is not so limited and the biasing member 178 may be a variety of different types of biasing members. FIGS. 5 and 6 illustrate the foldable knife 100 in the first configuration and FIG. 7 illustrates the foldable knife 100 in the second configuration.

The slider 172 is attached to the rod 176. The user may slide the slider 172 along the slot 174 of the locking mechanism 170. Movement of the slider 172 in a longitudinal direction of the first handle 110 simultaneously moves the rod 176 in the same direction as the slider 172. The rod 176 comprises a first end 176a and a second end 176b. The first end 176a is configured to engage with the biasing member 178. The second end 176b is configured to engage with the linkage 130.

The linkage 130 comprises a first slot 131 and a second slot 132. The first slot 131 is disposed 180 degrees from the second slot 132. The linkage 130 further comprises an arc shaped portion 133 that is disposed between the first slot 131 and the second slot 132. The linkage 130 further comprises a flat portion 134, opposite the arc shaped portion 133 and adjacent to the second slot 132. In embodiments where the linkage 130 is integral with the second handle 120, the flat portion 134 may be formed into the second end 122 of the second handle 120.

The locking mechanism 170 consists of two locking positions, a first locked position and a second locked position. In the first locked position, the rod 176 is received within the first slot 131 of the linkage 130. In other words, the first locked position of the locking mechanism 170 corresponds with the first configuration of the foldable knife 100. In the first locked position, the tip 144 and curved portion 145 of the blade 140 are concealed in the protective sleeve 160.

In the second locked position, the rod 176 is received within the second slot 132 of the linkage 130. In other words, the second locked position of the locking mechanism 170 corresponds with the second configuration of the foldable knife 100. In the second locked position, the blade 140 is exposed.

FIG. 5 specifically illustrates the foldable knife 100 in the first locked position. When the foldable knife 100 is in the first locked position, the second end 176b of the rod 176 engages with or is received within the first slot 131 of the linkage 130. The slider 172 is disposed in a first position. The user may slide the slider 172 away from the linkage 130 which simultaneously slides the rod 176 away from the linkage 130 to a second position of the slider 172 as illustrated in FIG. 6. Sliding the slider 172 from the first position to the second position compresses the biasing member 178. When the slider 172 is the second position, the locking mechanism 170 is unlocked and the user may rotate the second handle 120 relative to the first handle 110 approximately 180 degrees.

Once the second handle 120 begins to rotate, the user can release the slider 172 enabling the biasing member 178 to bias the second end 176b of the rod 176 to engage with the arc shaped portion 133. The second handle 120 continues to rotate until the second end 176b of the rod 176 is received into the second slot 132 that is 180 degrees from the first slot 131 as illustrated in FIG. 7. As illustrated in FIG. 7, the slider 172 returns to the first position.

As illustrated in FIGS. 1 and 2, the arc shaped portion 133 of the linkage 130 is disposed on a first side of the first handle 110 in the first configuration and as illustrated in FIGS. 8 and 9, the arc shaped portion 133 is disposed on a second side of the first handle 110 in the second configuration. The first side and the second side being opposite sides of the first handle 110.

When the second end 176b of the rod 176 is received into the second slot 132, the locking mechanism 170 is in the second locked position that corresponds with the second configuration of the foldable knife 100. In other words, the second handle 120 rotates about 180 degrees relative to the first handle 110 as the locking mechanism 170 transitions from the first locked position to the second locked position or the foldable knife 100 transition from the first configuration to the second configuration.

FIGS. 8 and 9 illustrate the foldable knife 100 in the second configuration and the locking mechanism 170 in the second locked position. As discussed above, the blade 140 in the second configuration is exposed and enables the user to use the blade 140.

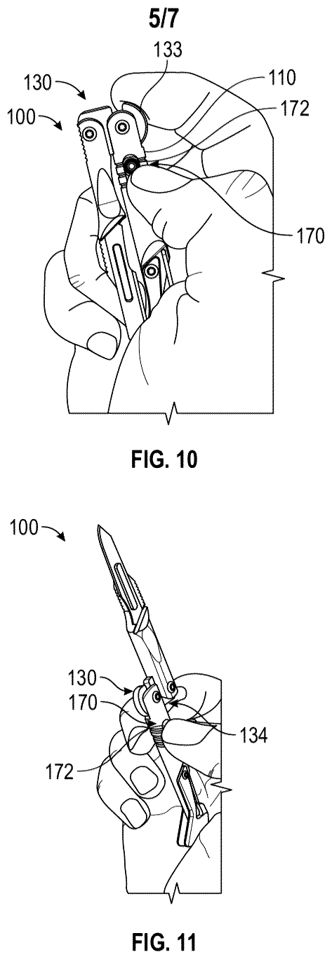

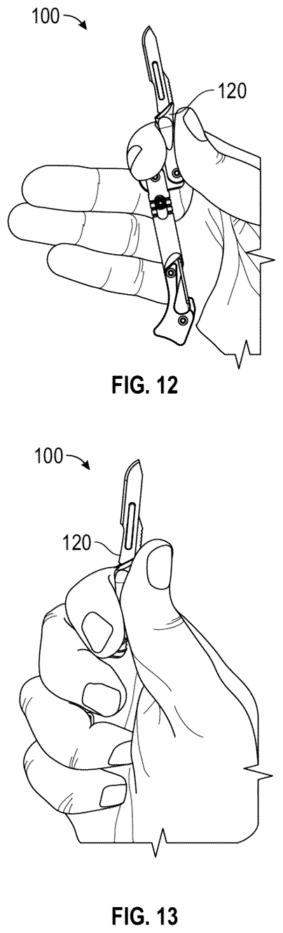

FIGS. 10 and 11 illustrate a process of the user transitioning the foldable knife 100 from the first configuration to the second configuration. For example, the user may grip the first handle 110 with their hand and grip the slider 172 with their thumb and their middle finger (or another suitable finger) to move the slider 172 from the first position to the second position to unlock the locking mechanism 170. Once the locking mechanism 170 is unlocked, the user may flick the arc shaped portion 133 of the linkage 130 with their index finger to rotate the second handle 120 to quickly transition the foldable knife 100 from the first configuration to the second configuration exposing the blade 140 as illustrated in FIG. 11. The user may release the slider 172 enabling the locking mechanism 170 to transition to the second locked position with the second end 176b of the rod 176 engaging with the second slot 132. In some circumstances, instead of flicking, the user may rotate the second handle 120 relative to the first handle 110 to the second configuration.

The user may also transition the foldable knife 100 from the second configuration back to the first configuration. For example, the user may grip the slider 172 with their thumb and their middle finger (or another suitable finger) to move the slider 172 from the first position to the second position to unlock the locking mechanism 170. Once the locking mechanism 170 is unlocked, the user may flick the flat portion 134 of the linkage 130 (or the second end 122 of the second handle 120) with their index finger to rotate the second handle 120 to quickly transition the foldable knife 100 from the second configuration to the first configuration and partially concealing the blade 140 as illustrated in FIG. 10. The user may release the slider 172 enabling the locking mechanism 170 to transition to the first locked position with the second end 176b of the rod 176 engaging with the first slot 131. In some circumstances, instead of flicking, the user may rotate the second handle 120 relative to the first handle 110 to position the foldable knife 100 to the first configuration.

FIGS. 12-14 illustrate various ways of gripping the foldable knife 100. However, the present disclosure is not limited to the illustrated ways of gripping the foldable knife 100 as alternative ways of gripping the foldable knife 100 are within the scope of the disclosure. FIG. 12 illustrates the user gripping the second handle 120 with their thumb and index finger (or another suitable finger). The user may grip the top of the second handle 120 with their thumb and the bottom of the second handle 120 with their index finger.

FIG. 13 illustrates another way of gripping the foldable knife 100. The user may grip the second handle 120 with their thumb and their index finger and the first handle 110 may be gripped with middle finger, ring finger, and the pinky.

Returning to FIGS. 8 and 9, the second handle 120 may comprise additional features for ease of holding and using the foldable knife 100. In some embodiments, the second handle 120 comprises a first finger groove 124 and a second finger groove 125. The first finger groove 124 is disposed on one side of the second handle 120 (see FIG. 8) and the second finger groove 125 is disposed on the opposite side of the second handle 120 (see FIG. 9). The second handle 120 may further comprise gripping portions 126 and 127. The first gripping portion 126 is disposed near the first end 121 of the second handle 120 and the second gripping portion 127 is disposed near the second end 122 of the second handle 120.

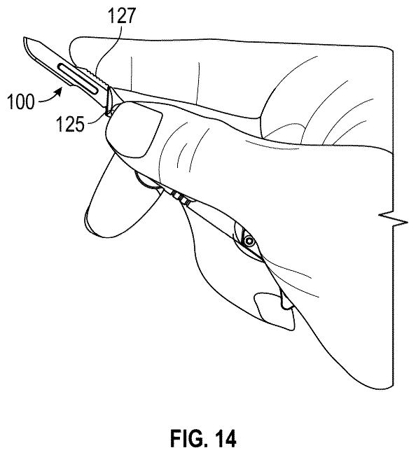

FIG. 14 illustrates another way of gripping the foldable knife 100. The user may grip the second handle 120 with the thumb in the first finger groove 124, the middle finger in the second finger groove 125, and the index finger gripping the second gripping portion 127. This way of gripping the foldable knife 100 may give control the user to precisely use the blade 140.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A foldable knife comprising:
a first handle;
a second handle pivotably coupled to the first handle;
a blade attached to the second handle and not to the first handle; and
a locking mechanism configured to selectively secure the foldable knife in a first configuration and a second configuration;
wherein the blade is partially concealed in the first configuration and the blade is exposed in the second configuration,
wherein in the first configuration, the first handle and the second handle are disposed in parallel planes; and
wherein in the second configuration, the first handle and the second handle are disposed in different parallel planes.

2. The foldable knife of claim 1, wherein in the first configuration, the second handle is disposed on a first longitudinal side of the first handle; and
wherein in the second configuration, the second handle is disposed on a second longitudinal side of the first handle, opposite the first longitudinal side of the first handle.

3. The foldable knife of claim 1, wherein in the first configuration, the first handle and the second handle overlap a longitudinal length of the second handle; and
wherein in the second configuration, the first handle and the second handle only overlap at a pivot connection between the first handle and the second handle.

4. The foldable knife of claim 1, wherein the blade is a scalpel.

5. The foldable knife of claim 4, wherein the scalpel is removably attachable to the second handle.

6. The foldable knife of claim 5, wherein the second handle comprises titanium and a portion of the second handle contacting the scalpel comprises a material other than titanium.

7. The foldable knife of claim 1, wherein the first handle comprises a protective sleeve that is configured to receive a tip of the blade in the first configuration.

8. The foldable knife of claim 7, wherein the protective sleeve comprises a pair of opposing guards forming a slot in between, each guard of the pair of opposing guards are attached to opposite sides of the first handle; and
wherein the tip of the blade is configured to be received within the slot in the first configuration.

9. A foldable knife comprising:
a first handle;
a second handle;
a linkage that pivotably couples the first handle to the second handle, wherein the linkage is rigidly attached to the second handle;
a blade attached to the second handle; and
a biasing locking mechanism configured to engage with the linkage to selectively secure the foldable knife in a first configuration and a second configuration;
wherein in the first configuration, a tip of the blade is disposed within a protective sleeve attached to the first handle; and
wherein in the second configuration, the tip of the blade is exposed.

10. The foldable knife of claim 9, wherein the biasing locking mechanism comprises a rod and a biasing member disposed within the first handle; and
wherein the biasing member biases the rod to engage with the linkage.

11. The foldable knife of claim 10, wherein the locking mechanism further comprises a slider attached to the rod; and
wherein movement of the slider in a longitudinal direction of the first handle simultaneously moves the rod in the same direction as the slider.

12. The foldable knife of claim 10, wherein the linkage comprises a first slot and a second slot;
wherein the first slot is disposed 180 degrees from the second slot; and
wherein the first slot is configured to receive the rod in the first configuration and the second slot is configured to receive the rod in the second configuration.

13. The foldable knife of claim 12, wherein the linkage comprises an arc shaped portion that is disposed between the first slot and the second slot.

14. The foldable knife of claim 13, wherein the arc shaped portion of the linkage is disposed on opposite sides of the first handle in the first configuration and in the second configuration.

15. The foldable knife of claim 9, wherein the linkage is integral with the second handle.

16. A foldable knife comprising:
a first handle having a first end and a second end;
a second handle having a first end and a second end, the second handle pivotably coupled to the first handle at a second end of the first handle and a first end of the second handle; and
a blade attached to the second end of the second handle, the blade having a cutting element;
wherein the foldable knife is moveable between a first locked position and a second locked position;
wherein the foldable knife in the first locked position has the cutting element of the blade oriented towards the first handle; and
wherein the foldable knife in the second locked position has a stepped shape, a longitudinal distance from the second end of second handle to the first end of the first handle in the second locked position is greater than the longitudinal distance from the second end of second handle to the first end of the first handle in the first locked position.

17. The foldable knife of claim 16, wherein the blade is not directly attached to the first handle.

18. The foldable knife of claim 16, further comprising a protective sleeve attached to the first handle that is configured to receive a tip of the blade in the first locked position.

19. The foldable knife of claim 18, wherein a straight portion of the cutting element of the blade is not disposed within the protective sleeve in the first locked position.

20. The foldable knife of claim 16, wherein a width of the blade is thinner than a width of the first handle; and
wherein the width of the blade is thinner than a width of the second handle.
